(19) United States
(12) Patent Application Publication
(10) Pub. No.: US 2026/0151460 A1
(43) Pub. Date: Jun. 4, 2026

(54) TREATMENT OF POST-BARIATRIC HYPOGLYCEMIA

(71) Applicant: Amylyx Pharmaceuticals, Inc., Cambridge, MA (US)

(72) Inventors: Robert Ryan Miller, Seattle, WA (US); Colleen M. Craig, Burlingame, CA (US)

(21) Appl. No.: 19/420,848

(22) Filed: Dec. 16, 2025

Related U.S. Application Data
(63) Continuation of application No. PCT/US2025/057590, filed on Dec. 2, 2025.
(60) Provisional application No. 63/726,872, filed on Dec. 2, 2024.

(51) Int. Cl. A61K 38/26; A61K 9/00; A61K 47/12; A61K 47/26; A61P 3/08

(52) U.S. Cl. CPC A61K 38/26; A61K 9/0019; A61K 47/12; A61K 47/26; A61P 3/08

(57) ABSTRACT

Provided herein are compositions and methods for treating post-bariatric hypoglycemia (PBH) in a subject in need thereof, including administration of an effective amount of avexitide.

### CROSS-REFERENCE TO RELATED APPLICATION

The present application is a continuation of and claims benefit under 35 U.S.C. § 120 from PCT Application No PCT/US2025/057590, filed on Dec. 2, 2025, which claims priority to U.S. Provisional Application No. 63/726,872, filed on Dec. 2, 2024. The entire contents of each of these earlier applications are herein incorporated by reference in their entirety.

### BACKGROUND

Insulin is a hormone secreted to control high blood glucose levels. Abnormal increases in insulin secretion can lead to hypoglycemia that can result in seizures, brain damage, and death. Glucagon-like peptide-1 (GLP-1) is a gastrointestinal hormone that is released postprandially from intestinal L-cells and binds to GLP-1 receptors on beta cells of the pancreas, thereby enhancing insulin release.

Hypoglycemia is a rare but serious complication of bariatric and gastric surgery, most commonly Roux-en-Y gastric bypass (RYGB), vertical sleeve gastrectomy (VSG) and total gastrectomy; this condition is referred to as post-bariatric hypoglycemia (PBH). PBH manifests as frequent, symptomatic, postprandial hypoglycemia that can lead to life-threatening neuroglycopenic consequences, such as loss of consciousness, seizures, cognitive dysfunction, and death. Quality of life can be severely diminished, and many patients cannot care for themselves or others, work, drive, or be left alone. As the number of bariatric surgeries to treat severe obesity has increased, so too has the number of individuals who experience PBH. Accordingly, there is a growing unmet need for a therapy that safely and effectively mitigates hyperinsulinemic hypoglycemia and PBH.

### SUMMARY

The present disclosure features formulations and methods for treating post-bariatric hypoglycemia (PBH) in a subject in need thereof. In some aspects, provided herein are methods of treating PBH in a subject in need thereof, the method comprising administering to the subject per day about 50 mg to about 150 mg of avexitide or a pharmaceutically acceptable salt thereof (e.g., avexitide acetate), wherein the method reduces a composite rate of Level 2 hypoglycemia events and Level 3 hypoglycemia events in the subject relative to a subject who has not received avexitide (e.g., baseline or placebo). In some embodiments, the subject has undergone bariatric surgery. In some embodiments, the subject has undergone bariatric surgery at least 12 months prior to administration. In some embodiments, the bariatric surgery is Roux-en-Y gastric bypass or vertical sleeve gastrectomy. In some embodiments, the subject has experienced hypoglycemic events. In some embodiments, the subject has experienced at least three hypoglycemic events during a 3-week window prior to administration. In some embodiments, at least two of the at least three hypoglycemic events are Level 2 or greater in severity. In some embodiments, at least one of the at least three hypoglycemic events is Level 3. In some embodiments, the subject has refractory PBH. In some embodiments, the method reduces hypoglycemic events relative to a 3-week window (e.g., the 3-week window with at least three hypoglycemic events as described above) prior to treatment. In some embodiments, the method reduces Level 2 hypoglycemia events in the subject. In some embodiments, the method reduces Level 2 hypoglycemia events in the subject by about 5% to about 100% (e.g., by about 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method reduces Level 2 hypoglycemia events as measured by self-monitoring of blood glucose (SMBG) and/or continued glucose monitoring (CGM). In some embodiments, the method reduces Level 3 hypoglycemia events in the subject. In some embodiments, the method reduces Level 3 hypoglycemia events in the subject by about 5% to about 100% (e.g., by about 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method reduces the composite rate of Level 2 hypoglycemia events and Level 3 hypoglycemia events in the subject by at least about 10%, 20%, 30%, 40%, 50%, 60%, 70%, 80%, or 90%. %). In some embodiments, the method reduces Level 1 hypoglycemia events in the subject. In some embodiments, the method reduces Level 1 hypoglycemia events in the subject by at least about 10%, 20%, 30%, 40%, 50%, 60%, 70%, 80%, or 90%. In some embodiments, the method reduces Level 1 hypoglycemia events, Level 2 hypoglycemia events, and Level 3 hypoglycemia events in the subject. In some embodiments, the method reduces the percent time with glucose levels less than about 70 mg/dL for the subject. In some embodiments, the method reduces the percent time with glucose levels less than about 70 mg/dL for the subject by at least about 5% to about 90% (e.g., by about 5%, 10%, 15%, 20%, 25%, 30%, 35%, 40%, 45%, 50%, 55%, 60%, 65%, 70%, 75%, 80%, 85%, or 90%). In some embodiments, the method reduces the percent time with glucose levels less than about 54 mg/dL for the subject. In some embodiments, the method reduces the percent time with glucose levels less than about 54 mg/dL for the subject by at least about 5% to about 90% (e.g., by about 5%, 10%, 15%, 20%, 25%, 30%, 35%, 40%, 45%, 50%, 55%, 60%, 65%, 70%, 75%, 80%, 85%, or 90%). In some embodiments, the method improves dietary liberalization in the subject. In some embodiments, the method includes administering about 80 mg to about 100 mg of the avexitide or the pharmaceutically acceptable salt thereof per day. In some embodiments, the avexitide or the pharmaceutically acceptable salt thereof is administered once per day. In some embodiments, the method includes administering about 90 mg of the avexitide or the pharmaceutically acceptable salt thereof once per day. In some embodiments, the avexitide or the pharmaceutically acceptable salt thereof is administered at least about 30 minutes to about 90 minutes before a meal.

In some embodiments, the avexitide or the pharmaceutically acceptable salt thereof (e.g., avexitide acetate) is administered in a formulation comprising about 90 mg/mL to about 110 mg/mL of the avexitide or the pharmaceutically acceptable salt thereof. In some embodiments, the avexitide or the pharmaceutically acceptable salt thereof is administered in a formulation comprising about 100 mg/mL of the avexitide or the pharmaceutically acceptable salt thereof. In some embodiments, the pharmaceutically acceptable salt of avexitide is avexitide acetate. In some embodiments, the avexitide acetate is a combination of avexitide monoacetate, avexitide diacetate, avexitide triacetate, avexitide tetraacetate, avexitide pentaacetate, and/or avexitide hexaacetate. In some embodiments, the avexitide acetate comprises primarily avexitide diacetate and/or avexitide triacetate. In some embodiments, the formulation comprises a physiologically acceptable buffer having a pH of about 5.0 to about 6.0. In some embodiments, the formulation comprises a physiologically acceptable buffer having a pH of about 5.2 to about 5.7. In some embodiments, the formulation comprises a physiologically acceptable buffer having a pH of about 5.4 or 5.5. In some embodiments, the physiologically acceptable buffer comprises sodium acetate trihydrate. In some embodiments, the sodium acetate trihydrate is present in the formulation at a concentration of about 1 mg/mL to about 1.5 mg/mL. In some embodiments, the sodium acetate trihydrate is present in the formulation at a concentration of about 1.36 mg/mL. In some embodiments, the formulation comprises a tonicity modifier. In some embodiments, the tonicity modifier is mannitol. In some embodiments, the mannitol is present in the formulation at a concentration of about 18 mg/mL to about 25 mg/mL. In some embodiments, the mannitol is present in the formulation at a concentration of about 19 mg/mL. In some embodiments, the formulation has an osmolality of about 260 mOsm/kg to about 380 mOsm/kg. In some embodiments, the formulation has an osmolality of at least 280 mOsm/kg. In some embodiments, the formulation has an osmolality of about 300 mOsm/kg to about 380 mOsm/kg. In some embodiments, the formulation has an osmolality of less than about 330 mOsm/kg. In some embodiments, the formulation has an osmolality of about 330 mOsm/kg (e.g., about 325, 326, 327, 328, 329, 330, 331, 332, 333, 334, or 335 mOsm/kg). In some embodiments, the avexitide or the pharmaceutically acceptable salt thereof is administered in a total volume of about 0.5 mL to about 1.5 mL. In some embodiments, the avexitide or the pharmaceutically acceptable salt thereof is administered in a total volume of about 0.9 mL.

In another aspect, provided herein are methods of treating PBH in a subject in need thereof, wherein method comprises administering to the subject a formulation comprising avexitide or a pharmaceutically acceptable salt thereof (e.g., avexitide acetate), wherein the avexitide or the pharmaceutically acceptable salt thereof is administered at a dose of about 50 mg to about 150 mg, wherein the formulation comprises the avexitide or the pharmaceutically acceptable salt thereof at a concentration between about 90 mg/mL and about 110 mg/mL, sodium acetate trihydrate at a concentration between about 1 mg/mL and about 1.5 mg/mL, and mannitol at a concentration between about 18 mg/mL and about 25 mg/mL, wherein the formulation has a pH of about 5.2 to about 5.7, and wherein the method reduces a composite rate of Level 2 hypoglycemia events and Level 3 hypoglycemia events in the subject relative to a subject who has not received avexitide (e.g., baseline or placebo). In some embodiments, the subject has undergone bariatric surgery at least 12 months prior to administration. In some embodiments, the bariatric surgery is Roux-en-Y gastric bypass or vertical sleeve gastrectomy. In some embodiments, the subject has experienced at least three hypoglycemic events during a 3-week window prior to administration. In some embodiments, at least two of the at least three hypoglycemic events are Level 2 or greater in severity, and wherein at least one of the at least three hypoglycemic events is Level 3. In some embodiments, the subject has refractory PBH. In some embodiments, Level 2 hypoglycemia events are measured by SMBG and/or CGM. In some embodiments, the method reduces the composite rate of Level 2 hypoglycemia events and Level 3 hypoglycemia events in the subject relative to baseline by at least about 10% (e.g., by at least about 10%, 20%, 30%, 40%, 50%, 60%, 70%, 80%, or 90%). In some embodiments, the method further reduces Level 1 hypoglycemia events in the subject relative to baseline and/or dietary liberalization in the subject relative to baseline. In some embodiments, the method reduces Level 1 hypoglycemia events in the subject by at least about 10%, 20%, 30%, 40%, 50%, 60%, 70%, 80%, or 90%. In some embodiments, the method reduces the percent time with glucose levels less than about 70 mg/dL for the subject relative to baseline. In some embodiments, the method reduces the percent time with glucose levels less than about 70 mg/dL for the subject by at least about 5% to about 90% (e.g., by about 5%, 10%, 15%, 20%, 25%, 30%, 35%, 40%, 45%, 50%, 55%, 60%, 65%, 70%, 75%, 80%, 85%, or 90%). In some embodiments, the method reduces the percent time with glucose levels less than about 54 mg/dL for the subject relative to baseline. In some embodiments, the method reduces the percent time with glucose levels less than about 54 mg/dL for the subject by at least about 5% to about 90% (e.g., by about 5%, 10%, 15%, 20%, 25%, 30%, 35%, 40%, 45%, 50%, 55%, 60%, 65%, 70%, 75%, 80%, 85%, or 90%). In some embodiments, the method comprises administering about 80 mg to about 100 mg of the avexitide or the pharmaceutically acceptable salt thereof per day. In some embodiments, the avexitide or the pharmaceutically acceptable salt thereof is administered once per day at a dose of about 90 mg. In some embodiments, the avexitide or the pharmaceutically acceptable salt thereof is administered at least about 30 minutes to about 90 minutes before a meal. In some embodiments, the avexitide or the pharmaceutically acceptable salt thereof is present in the formulation at a concentration of about 100 mg/mL. In some embodiments, the pharmaceutically acceptable salt of avexitide is avexitide acetate. In some embodiments, the avexitide acetate is a combination of avexitide monoacetate, avexitide diacetate, avexitide triacetate, avexitide tetraacetate, avexitide pentaacetate, and/or avexitide hexaacetate. In some embodiments, the avexitide acetate comprises primarily avexitide diacetate and/or avexitide triacetate. In some embodiments, the formulation comprises a pH of about 5.4 or about 5.5. In some embodiments, the sodium acetate trihydrate is present in the formulation at a concentration of about 1.36 mg/mL. In some embodiments, the mannitol is present in the formulation at a concentration of about 19 mg/mL. In some embodiments, the formulation has an osmolality of about 260 mOsm/kg to about 380 mOsm/kg. In some embodiments, the formulation has an osmolality of about 280 mOsm/kg to about 380 mOsm/kg. In some embodiments, the formulation has an osmolality of about 300 mOsm/kg to about 380 mOsm/kg. In some embodiments, the formulation has an osmolality of less than about 300 mOsm/kg. In some embodiments, the formulation has an osmolality of about 330 mOsm/kg (e.g., about 325, 326, 327, 328, 329, 330, 331, 332, 333, 334, or 335 mOsm/kg). In some embodiments, the avexitide or the pharmaceutically acceptable salt thereof is administered subcutaneously in a total volume of about 0.5 mL to about 1.5 mL. In some embodiments, the avexitide or the pharmaceutically acceptable salt thereof is administered subcutaneously in a total volume of about 0.9 mL.

In another aspect, provided herein is a formulation comprising avexitide or a pharmaceutically acceptable salt thereof (e.g., avexitide acetate) at a concentration between about 90 mg/mL and 110 mg/mL, sodium acetate trihydrate at a concentration between about 1 mg/mL and about 1.5 mg/mL, and mannitol at a concentration between about 18 mg/mL and about 25 mg/mL, wherein the formulation has a pH of about 5.2 to about 5.7, and wherein the formulation has an osmolality of about 260 mOsm/kg to about 380 mOsm/kg. In some embodiments, the formulation comprises the avexitide or the pharmaceutically acceptable salt thereof at a concentration of about 100 mg/mL. In some embodiments, the pharmaceutically acceptable salt of avexitide is avexitide acetate. In some embodiments, the avexitide acetate is a combination of avexitide monoacetate, avexitide diacetate, avexitide triacetate, avexitide tetraacetate, avexitide pentaacetate, and/or avexitide hexaacetate. In some embodiments, the avexitide acetate comprises primarily avexitide diacetate and/or avexitide triacetate. In some embodiments, the formulation comprises the sodium acetate trihydrate at a concentration of about 1.36 mg/mL. In some embodiments, the formulation comprises the mannitol at a concentration of about 19 mg/mL. In some embodiments, the formulation has a pH of about 5.4 or about 5.5. In some embodiments, the formulation has an osmolality of about 280 mOsm/kg to about 380 mOsm/kg. In some embodiments, the formulation has an osmolality of about 300 mOsm/kg to about 380 mOsm/kg. In some embodiments, the formulation has an osmolality of less than about 300 mOsm/kg. In some embodiments, the formulation has an osmolality of about 330 mOsm/kg (e.g., about 325, 326, 327, 328, 329, 330, 331, 332, 333, 334, or 335 mOsm/kg). In some embodiments, the formulation is used in a method of treating PBH in a subject in need thereof, wherein the formulation comprises administering to the subject the formulation, and wherein the method reduces a composite rate of Level 2 hypoglycemia events and Level 3 hypoglycemia events in the subject relative to a subject who has not received avexitide (e.g., baseline or placebo). In some embodiments, the method comprises administering the formulation at a dose of 50 mg to 150 mg of the avexitide or the pharmaceutically acceptable salt thereof. In some embodiments, the subject has undergone bariatric surgery at least 12 months prior to administration. In some embodiments, the bariatric surgery is Roux-en-Y gastric bypass or vertical sleeve gastrectomy. In some embodiments, the subject has experienced at least three hypoglycemic events during a 3-week window prior to administration. In some embodiments, at least two of the at least three hypoglycemic events are Level 2 or greater in severity, and wherein at least one of the at least three hypoglycemic events is Level 3. In some embodiments, the subject has refractory PBH. In some embodiments, Level 2 hypoglycemia events are measured by SMBG and/or CGM. In some embodiments, the method reduces the composite rate of Level 2 hypoglycemia events and Level 3 hypoglycemia events in the subject relative to baseline by at least about 10% (e.g., by at least about 10%, 20%, 30%, 40%, 50%, 60%, 70%, 80%, or 90%). In some embodiments, the method further reduces Level 1 hypoglycemia events in the subject relative to baseline and/or dietary liberalization in the subject relative to baseline. In some embodiments, the method reduces Level 1 hypoglycemia events in the subject by at least about 10%, 20%, 30%, 40%, 50%, 60%, 70%, 80%, or 90%. In some embodiments, the method reduces the percent time with glucose levels less than about 70 mg/dL for the subject relative to baseline. In some embodiments, the method reduces the percent time with glucose levels less than about 70 mg/dL for the subject by at least about 5% to about 90% (e.g., by about 5%, 10%, 15%, 20%, 25%, 30%, 35%, 40%, 45%, 50%, 55%, 60%, 65%, 70%, 75%, 80%, 85%, or 90%). In some embodiments, the method reduces the percent time with glucose levels less than about 54 mg/dL for the subject relative to baseline. In some embodiments, the method reduces the percent time with glucose levels less than about 54 mg/dL for the subject by at least about 5% to about 90% (e.g., by about 5%10%, 15%, 20%, 25%, 30%, 35%, 40%, 45%, 50%, 55%, 60%, 65%, 70%, 75%, 80%, 85%, or 90%). In some embodiments, the method comprises administering about 80 mg to about 100 mg of the avexitide or the pharmaceutically acceptable salt thereof per day. In some embodiments, the avexitide or the pharmaceutically acceptable salt thereof is administered once per day at a dose of about 90 mg. In some embodiments, the avexitide or the pharmaceutically acceptable salt thereof is administered at least about 30 minutes to about 90 minutes before a meal. In some embodiments, the avexitide or the pharmaceutically acceptable salt thereof is administered subcutaneously in a total volume of about 0.5 mL to about 1.5 mL. In some embodiments, the avexitide or the pharmaceutically acceptable salt thereof is administered subcutaneously in a total volume of about 0.9 mL.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIGURE, and from the claims.

### BRIEF DESCRIPTION OF THE DRAWINGS

FIG. 1 is a schematic of the study design.

### DETAILED DESCRIPTION

The present disclosure is based, at least in part, on the insight that the methods and compositions provided herein are particularly useful for treating or preventing post-bariatric hypoglycemia (PBH). The disclosure encompasses methods of administering avexitide to a subject in need thereof for treating or preventing PBH. Also provided are compositions of avexitide useful for treatment of PBH. The compositions and methods provided herein are able to reduce signs and symptoms of PBH, including, for example, reducing the rate at which hypoglycemic events (e.g., Level 1, 2, or 3 hypoglycemic events) occur, increasing the percent time in target glucose range (TIR)/reducing the percent time below target glucose range (TBR), and/or improving dietary liberalization.

### Avexitide

Avexitide is a 31-amino acid peptide that selectively targets and blocks GLP-1 receptors, normalizing insulin secretion by the pancreas, and thereby reducing postprandial hypoglycemia. Avexitide treatment reduces not only post-prandial (protein-induced) hypoglycemia but also fasting hyperinsulinemic hypoglycemia.

Avexitide, also referred to as “exendin (9-39),” has an empirical formula of C₁₄₉H₂₃₄N₄₀O₄₇S and a molecular weight of 3369.8 Daltons. Avexitide comprises residues 9-39 of the GLP-1 receptor agonist exendin-4 and is a GLP-1 receptor antagonist. See Montrose-Rafizadeh et al., Journal of Biological Chemistry, 272:21201-21206 (1997). The amino acid sequence for avexitide is shown as follows: H-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2 (SEQ ID NO: 1). Avexitide has a predicted isoelectric point of 4.69 and has a net charge of −1 at pH 6 that increases to a net charge of +4 at pH 3.0. As used herein, the term “avexitide” also encompasses pharmaceutically acceptable salts of avexitide (exendin (9-39)), including but not limited to acetate, sulfate, hydrochloride, phosphate, sulfamate, citrate, lactate, tartrate, methanesulfonate, ethanesulfonate, benzenesulfonate, p-toluenesulfonate, cyclohexylsulfamate and quinate salts. In some embodiments, avexitide is in the form of an acetate or trifluoroacetate salt. In some embodiments, avexitide is in the form of avexitide acetate. Avexitide (exendin (9-39)) and pharmaceutically acceptable salts thereof are commercially available.

### Therapeutic Methods

In one aspect, provided herein are methods of treating or preventing post-bariatric hypoglycemia (PBH) by administering to a subject a composition comprising avexitide. As described herein, in some embodiments, treatment with avexitide prevents or reduces one or more symptoms, metabolic outcomes, and/or clinical outcomes of post-bariatric hypoglycemia.

### Patient Population

In some embodiments, a subject to be treated according to the methods described herein is a subject having post-bariatric hypoglycemia (PBH) who has previously had a bariatric and/or related metabolic procedure. Bariatric and/or related metabolic procedures include, but are not limited to, Roux-en-Y Gastric Bypass, Vertical Sleeve Gastrectomy, placement of an endosleeve device, such as the EndoBarrier Gastrointestinal Liner System, also called an “endoluminal liner,” duodenal mucosal resurfacing (also referred to as duodenal ablation), biliopancreatic diversion with duodenal switch, partial bypass of the duodenum, involving duodeno-ileal or duodeno-jejunal anastomosis, vagal nerve blockade, and pyloroplasty. The subject may in some cases have had a bariatric and/or related metabolic procedure at least about one month to about five years prior to the onset of treatment. For example, the subject may have had a bariatric and/or related metabolic procedure at least about one year prior to the onset of treatment.

A bariatric procedure (e.g., bariatric surgery) can involve any of the following: partially or completely bypassing the duodenum and/or decreasing nutrient exposure to the duodenum, increasing the rapidity of nutrient transit to the lower part of the intestines (often specifically the ileum), and/or otherwise increasing ileal nutrient exposure. Bariatric surgery may be intended for weight loss or metabolic benefit (such as resolution of diabetes), or both. Such weight loss or metabolic procedures, referred to herein as “bariatric procedures,” may enhance secretion of GLP-1 from the distal small intestine, especially the ileum, leading to elevated insulin secretion, and in some subjects, hypoglycemia. In some embodiments, the subject may be referred to as a “post-bariatric surgery subject,” “subject with PBH,” or “patient.”

Subjects to be treated according to the methods described herein include those that have previously had a Roux-en-Y Gastric Bypass. For example, the subject may have had a Roux-en-Y Gastric Bypass at least one year prior to the onset of treatment.

In some embodiments, the subject to be treated has previously had a related procedure. As a non-limiting example, in one embodiment, the subject to be treated has previously had a non-bariatric surgical procedure involving the gastrointestinal system (including but not limited to esophagectomy, for example for treatment of esophageal cancer, Nissen Fundoplication, for example for treatment of gastroesophageal reflux, or gastrectomy, for example for treatment or prevention or treatment of gastric cancer) and so may be referred to herein as “post gastrointestinal surgery.”

Subjects that can be treated using the compositions and methods provided herein, including, e.g., subjects with PBH, can be identified by any suitable methods.

In some embodiments, PBH is diagnosed by the presence of Whipple's triad in a post-bariatric patient, which has the following criteria: (1) the occurrence of hypoglycemic symptoms (e.g., neuroglycopenic symptoms such as behavioral changes, confusion or impaired cognitive function, seizure, loss of consciousness); (2) documented low blood glucose level at the time of the symptoms (e.g., <54 mg/dL; <70 mg/dL); and (3) resolution of the symptoms after blood glucose is raised (e.g., within a few minutes). In some embodiments, PBH is diagnosed using a positive provocative test, e.g., an oral glucose tolerance test (OGTT) or a mixed meal tolerance test (MMTT). In some embodiments, PBH is diagnosed in a post-bariatric patient as the presence of symptomatic postprandial hypoglycemia associated with inappropriately elevated insulin (≥3 μU/mL) or C-peptide (>0.6 ng/mL) at the time of hypoglycemia (<54 mg/dL glucose). Diagnostic criteria for PBH is also disclosed in Salehi et al., The Journal of Clinical Endocrinology & Metabolism, 2018, 103:2815-2826, which is incorporated by reference herein in its entirety.

The compositions and methods provided herein are also useful for treating subjects with refractory PBH. As used herein, a subject or patient having “refractory PBH” refers to a PBH patient who is undergoing conventional treatment, e.g., dietary modification, or the use of rescue glucagon but who still experiences (1) hypoglycemia that interferes with the patient's daily activities, and/or (2) severe hypoglycemic events.

In some cases, the subjects to be treated with the compositions and methods provided herein have recurrent hypoglycemia. For example, the subject may have two or more hypoglycemic events over the course of several weeks. In some cases, the subject experiences at least 2, 3 or 4 hypoglycemic events during a 2-week or 3-week period. In some cases, the subject experiences at least 3 hypoglycemia events during a 3-week period. The hypoglycemic events can be discrete events that occur more than a certain amount of time apart. For example, discrete hypoglycemia events can be those that occur at least 60 minutes or more apart.

In some cases, the hypoglycemic events is characterized by glucose levels of <54 mg/dL (3.0 mmol/L), for example, as measured by SMBG (self-monitoring of blood glucose). Any suitable SMBG device can be used for this purpose. Such events can also be referred to as Level 2 hypoglycemic events. In some cases, SMBG measurements that are <54 mg/dL (<3.0 mmol/L) within 60 minutes of the first instance of <54 mg/dL are considered 1 discrete event. In some cases, the subject experiences at least 1, 2, 3, or 4 Level 2 hypoglycemic event(s) (e.g., discrete hypoglycemic events) during a 2-week or 3-week time window. If SMBG is not obtained at the time of symptoms, or if the patient does not experience symptoms due to hypoglycemia unawareness, hypoglycemic events (e.g., Level 2 hypoglycemic events) may be documented based on a corresponding continuous glucose monitor (CGM) measurement of ≤54 and/or neurological recovery following the return of glucose levels to normal.

In some cases, the hypoglycemic event is characterized by altered mental and/or physical functioning that requires assistance from another person for recovery (regardless of whether a patient actually receives external assistance). Such events can also be referred to as Level 3 hypoglycemic events. In some cases, the subject experiences at least 1, 2, 3, or 4 Level 3 hypoglycemic events (e.g., discrete hypoglycemic events) during a 2-week or 3-week time window. In some cases, if a Level 3 hypoglycemic event occurred within the same 60-minute window of the Level 2 hypoglycemic event, they are counted as 1 discrete event. In some cases, the categorization of a Level 3 hypoglycemic event is adjudicated by an independent review body.

In some cases, the provided methods are useful for treating subjects who are at risk for developing PBH, for example, subjects who have had a bariatric procedure (e.g., a Roux-en-Y Gastric Bypass) but who have not displayed symptoms of PBH, or subjects who have had a related metabolic procedure but who have not displayed symptoms of PBH.

In some cases, the subjects treated with the compositions and methods provided herein are also subject to dietary management before, during and/or after treatment. For example, the subjects may follow on or more of the following: (1) limiting dietary carbohydrates to no more than 30 g per meal or 15 g per snack (See, e.g. Kellogg et al., Surg Obes Relat Dis. 2008 July-August; 4(4):492-9; Bantle et al., Obes Surg. 2007 May; 17(5):592-4; Botros et al., Obes Surg. 2014 November; 24(11):1850-5; van Meijeren et al., Surg Obes Relat Dis. 2017 March; 13(3):404-410; Suhl et al., Surg Obes Relat Dis. 2017 May; 13(5):888-896), (2) avoiding simple, high-glycemic index carbohydrates, (3) maintaining adequate protein intake (See, e.g., Heber et al., J Clin Endocrinol Metab. 2010 November; 95(11):4823-43; Moize et al., Obes Surg. 2010 August; 20(8):1133-41; Mechanick et al., American Association of Clinical Endocrinologists; Obesity Society; American Society for Metabolic & Bariatric Surgery. American Association of Clinical Endocrinologists, The Obesity Society, and American Society for Metabolic & Bariatric Surgery medical guidelines for clinical practice for the perioperative nutritional, metabolic, and nonsurgical support of the bariatric surgery patient. Obesity (Silver Spring). 2009 April; 17 Suppl 1:S1-70, v. Erratum in: Obesity (Silver Spring). 2010 March; 18(3):649; Suhl et al., Surg Obes Relat Dis. 2017 May; 13(5):888-896); (4) avoiding consumption of meals during the late evening; (5) restricting use of alcohol to no more than 1 drink per day; and (6) maintaining post-bariatric vitamin and mineral supplementation (See, e.g., Heber et al., J Clin Endocrinol Metab. 2010 November; 95(11):4823-43; Moize et al., Obes Surg. 2010 August; 20(8):1133-41).

### Treatment of Post-Bariatric Hypoglycemia

Methods described in the present disclosure can include treatment of PBH per se, as well as treatment for one or more symptoms of PBH. “Treating” PBH does not require 100% abolition of the disease or disease symptoms in the subject. Any relief or reduction in the severity of symptoms or features of the disease is contemplated. The disclosure contemplates any degree of alleviation of one or more signs or symptoms of the disease.

The treatment provided in the present disclosure can be initiated at any stage during disease progression. For example, treatment can be initiated prior to onset of symptoms, at symptom onset or immediately following detection of PBH symptoms, or upon observation of any one or more symptoms that would lead a skilled practitioner to suspect that the subject may be developing PBH. Treatment can also be initiated at later stages.

Treatment methods can include a single administration, multiple administrations, and repeating administration as required for the prophylaxis or treatment of PBH, or at least one symptom of PBH. The duration of prophylaxis treatment can be a single dosage or the treatment may continue (e.g., multiple dosages), e.g., for years or indefinitely for the lifespan of the subject. For example, a subject at risk for developing PBH may be treated with the methods provided herein for days, weeks, months, or years so as to prevent the disease from occurring or progressing.

The terms “administer,” “administering,” or “administration” as used herein refer to providing compositions described herein to a subject using any art-known method, e.g., ingesting, injecting, implanting, absorbing, or inhaling, the drug, regardless of form. The methods provided herein include administration of an effective amount of a composition disclosed herein to achieve the desired or stated effect. Specific dosage and treatment regimens for any particular subject will depend upon a variety of factors, including the activity of the specific composition employed, the age, body weight, general health status, sex, diet, time of administration, rate of excretion, drug combination, the severity and course of the disease, condition or symptoms, the subject's disposition to the disease, condition or symptoms, and the judgment of the treating physician.

The methods provided herein can also include assessing a level of disease in the subject prior to treatment, during treatment, and/or after treatment. For example, following administration of a composition described herein, the subject can be evaluated to detect, assess, or determine their level of disease. In some embodiments, treatment can continue until a change (e.g., reduction) in the level of disease in the subject is detected.

Upon improvement of a patient's condition (e.g., a change (e.g., decrease) in the level of disease in the subject), a maintenance dose of a composition of this disclosure may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

In some embodiments, the methods provided herein reduce hypoglycemia events experienced by the subject. The provided methods can, for example, reduce the number of hypoglycemic events experienced by the subject within a certain time frame (e.g., within a week, 2 weeks, 3 weeks, or a month). The provided methods can, for example, reduce the rate of Level 1, Level 2, and/or Level 3 hypoglycemia events as described herein.

In some cases, the methods reduce Level 2 hypoglycemia events experienced by the subject (e.g., as measure by SMBG or CGM). For example, the provided methods can reduce the number of Level 2 hypoglycemic events experienced by the subject over a certain period of time, e.g., relative to a subject who has not received the treatment or relative to the subject pre-treatment. As described herein, in some cases, a Level 2 hypoglycemia event involves the subject's glucose level being <54 mg/dL (3.0 mmol/L), for example, as measured by SMBG. In some cases, the treatments provided herein reduce Level 2 hypoglycemia events by about 5% to about 100% (e.g., about 10% to about 80%, about 20% to about 70%, about 30% to about 60%, about 40% to about 50%, or about 50% to about 60%) relative to placebo or to baseline.

In some cases, the methods reduce Level 3 hypoglycemia events experienced by the subject. For example, the provided methods can reduce the number of Level 3 hypoglycemic events experienced by the subject over a certain period of time, e.g., relative to a subject who has not received the treatment or relative to the subject pre-treatment. In some cases, a Level 3 hypoglycemia event involves altered mental and/or physical functioning that requires assistance from another person for recovery (regardless of whether a patient actually receives external assistance). In some cases, a Level 3 hypoglycemia event is adjudicated by an evaluator based on a classification criteria, e.g., per American Diabetes Association [ADA], or European Association for the Study of Diabetes [EASD]. In some cases, the treatments provided herein reduce Level 3 hypoglycemia events by about 5% to about 100% (e.g., about 10% to about 80%, about 20% to about 70%, about 20% to about 60%, about 30% to about 70%, or about 40% to about 70%) relative to placebo or to baseline.

In some embodiments, the methods provided herein reduce the composite Level 2 hypoglycemia events and Level 3 hypoglycemia events experienced by the subject, e.g., relative to a subject who did not receive the treatment or relative to the subject pre-treatment

The methods provided herein can also reduce one or more of the following: Time below target glucose range (TBR) Level 2 (e.g., as measured by CGM); TBR Level 1 (e.g., as measured by CGM); Time in target glucose range (TIR) (e.g., as measured by CGM); Time above target glucose range (TAR) (e.g., as measured by CGM); Level 1 hypoglycemia (e.g., as measured by CGM or SMBG); Extended Level 1 hypoglycemia (e.g., as measured by CGM); or Extended Level 2 hypoglycemia (e.g., as measured by CGM).

In some cases, TBR Level 2 is determined by the percent time that the subject's glucose level is less than 54 mg/dL. The methods provided herein can, for example, reduce the percent time with glucose<54 mg/dL (e.g., without clinically relevant increases in percent time with glucose>250 mg/dL). The provided methods can, in some cases, reduce the percent time with glucose<54 mg/dL by about 5% to about 90% (e.g., about 10% to about 50%, about 20% to about 50%, about 20% to about 30%, or about 40% to about 50%).

In some cases, TBR level 1 is determined by the percent time that the subject's glucose level is less than 70 mg/dL (e.g., as measured by SMBG or CGM). The methods provided herein can, for example, reductions in percent time with glucose<70 mg/dL (e.g., without clinically relevant increases in percent time with glucose>250 mg/dL). The provided methods can, in some cases, reduce the percent time with glucose<70 mg/dL by about 5% to about 90% (e.g., about 10% to about 50%, about 20% to about 50%, about 20% to about 30%, or about 40% to about 50%). In some cases, Level 1 hypoglycemia is where the subject's glucose is <70 mg/dL (e.g., as measured by SMBG or CGM).

In some cases, extended level 1 hypoglycemia refers to level 1 hypoglycemia that lasts for an extended duration, for example, 60 minutes or longer. In some cases, extended level 2 hypoglycemia refers to level 2 hypoglycemia that lasts for an extended duration, for example, 60 minutes or longer.

The methods provided herein can also improve quality of life as measured by one or more assessments, including e.g., Clarke Hypoglycaemia Awareness Survey (CHAS), Gold Score, Hypoglycemia Fear Survey-II (HFS-II), Short-Form (36) Health Survey version 2 (SF-36 v2), EuroQOL 5 Dimension 5-Level questionnaire (EQ-5D-5L), and Functional Assessment of Chronic Illness Therapy—Fatigue scale

### (FACIT-Fatigue).

In some cases, upon treatment with the methods disclosed herein, the subjects experience dietary liberalization. Dietary liberalization can involve improved nutrition or improved nutritional intake and improved appetite. In some cases, dietary liberalization is estimated by the number of glucose spikes or excursions (e.g. those that persist for a certain amount of time). For example, dietary liberalization can be assessed based on the number of glucose excursions≥150 mg/dL, as measured by CGM.

Treatment endpoints and methods of measuring treatment endpoints are also described in the Examples section below, e.g., at Table 1.

In some embodiments, treatment reduces the number and/or severity of postprandial neuroglycopenic symptoms in the subject. In some embodiments, treatment improves postprandial glucose nadir. In some embodiments, treatment reduces postprandial insulin peak. In some embodiments, the treatment reduces the rate of hypoglycemia-induced CNS impairment. In some embodiments, the administration of avexitide provides clinically meaningful improvement in one or more of the rate of hypoglycemia induced CNS impairment, severe hypoglycemia, postprandial insulin peak, rate of severe hypoglycemia, or postprandial glucose nadir.

For example, in some embodiments, treatment with avexitide therapy results in a reduction in the number and/or severity of postprandial neuroglycopenic symptoms as compared to baseline (e.g., as compared to the number and/or severity of postprandial neuroglycopenic symptoms in the subject prior to the onset of treatment, e.g., as measured during MMTT). In some embodiments, treatment with avexitide therapy as disclosed herein results in an improvement in postprandial insulin response. For example, in some embodiments, treatment with avexitide therapy results in a decrease in peak postprandial insulin concentration as compared to baseline (e.g., as compared to the subject's peak postprandial insulin concentration prior to the onset of treatment, e.g., as measured during MMTT). In some embodiments, treatment with avexitide therapy as disclosed herein results in an improvement in the patient's rate of hypoglycemia and/or rate of severe hypoglycemia. For example, in some embodiments, treatment with avexitide therapy results in a reduction in the rate of hypoglycemia and/or a reduction in the rate of severe hypoglycemia as compared to baseline or placebo or other control (e.g., as compared to the subject's rate of hypoglycemia and/or rate of severe hypoglycemia, e.g., as measured by SMBG or CGM).

### Dosing and Administration

Generally, the methods described herein include administering a therapeutically effective amount of avexitide as described herein to a subject needing, or who has been determined to be in need of, such treatment. Avexitide as described herein can be administered to a subject by a variety of methods. For example, avexitide can be administered subcutaneously (including e.g., subcutaneous injections or subcutaneous infusions) or intravenously (including, e.g., as a bolus or by intravenous infusion). The route and/or mode of administration can also be tailored for the individual case. As provided herein, administration of avexitide encompasses self-administration.

As provided herein, avexitide can be administered at a total daily dose in the range of about 50 mg to about 150 mg (e.g., about 50 mg to about 130 mg, about 50 mg to about 120 mg, about 50 mg to about 100 mg, about 60 mg to about 140 mg, about 60 mg to about 120 mg, about 60 mg to about 110 mg, about 60 mg to about 100 mg, about 60 mg to about 90 mg, about 70 mg to about 140 mg, about 70 mg to about 120 mg, about 70 mg to about 110 mg, about 70 mg to about 100 mg, about 70 mg to about 90 mg, about 80 mg to about 140 mg, about 80 mg to about 120 mg, about 80 mg to about 110 mg, about 80 mg to about 100 mg, about 85 mg to about 95 mg, about 90 mg to about 120 mg, about 90 mg to about 110 mg, or about 90 mg to about 100 mg). In some embodiments, avexitide is administered at a total daily dose of about 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, or 120 mg. In some embodiments, avexitide is administered at a total daily dose of about 90 mg. For example, avexitide can be administered (e.g., subcutaneously administered) at an amount of 90 mg once daily.

In some embodiments, avexitide is administered once daily (“QD”). For example, any of the total daily doses of avexitide as described herein can be administered as a once daily administration. In some cases, avexitide is administered at an amount of about 40 mg to about 120 mg QD (e.g., about 40 mg to about 110 mg QD, about 40 mg to about 100 mg QD, about 40 mg to about 90 mg QD, about 50 mg to about 120 mg QD, about 50 mg to about 110 mg QD, about 50 mg to about 100 mg QD, about 50 mg to about 90 mg QD, about 60 mg to about 120 mg QD, about 60 mg to about 110 mg QD, about 60 mg to about 100 mg QD, about 60 mg to about 90 mg QD, about 70 mg to about 120 mg QD, about 70 mg to about 110 mg QD, about 70 mg to about 100 mg QD, about 70 mg to about 90 mg QD, about 80 mg to about 120 mg QD, about 80 mg to about 110 mg QD, about 80 mg to about 100 mg QD, about 80 mg to about 90 mg QD, about 90 mg to about 120 mg QD, about 90 mg to about 110 mg QD, or about 90 mg to about 100 mg QD). In some embodiments, avexitide is administered at an amount of about 40 mg QD, 45 mg QD, 50 mg QD, 55 mg QD, 60 mg QD, 65 mg QD, 70 mg QD, 75 mg QD, 80 mg QD, 85 mg QD, 90 mg QD, 95 mg QD, 100 mg QD, 105 mg QD, 110 mg QD, 115 mg QD, or 120 mg QD.

QD doses can be administered (for example, subcutaneously administered) at about 24-hour intervals (for example, 7 a.m. on successive days). However, shorter (for example, about 8 a.m. and about 6 a.m. on successive days) or longer (for example, about 7 a.m. and about 9 a.m. on successive days) intervals between administration are possible provided the administrations are at least about 18 hours apart. In some cases, the administrations are at least about 18 hours, about 19 hours, about 20 hours, about 21 hours, about 22 hours, about 23 hours, or 24 hours apart. In some cases, the administrations are not more than about 30 hours apart.

Avexitide can also be administered multiple times daily, including, e.g., twice daily (“BID”) or three times daily (“TID”). For example, any of the total daily doses of avexitide as described herein can be administered as a twice daily administration. Avexitide can be administered at specific points in the day or schedule of a subject, for example, morning, afternoon, evening, night, before or during or after meals, or before bedtime. In some embodiments, the formulation is administered about once every 12 hours. In some embodiments BID doses are administered at about 12-hour intervals (for example, about 7 a.m. and about 7 p.m.). However, shorter (for example, about 8 a.m. and about 6 p.m.) or longer (for example, about 7 a.m. and about 10 p.m.) intervals between administrations are possible. In some embodiments, the administrations are at least about 4 hours, about 6 hours, about 7 hours, about 8 hours, about 9 hours, about 10 hours or about 11 hours apart. In some embodiments, the administrations are not more than about 15 hours apart.

In some cases, avexitide is administered less than once daily, including, e.g., once every two days, once every three days, or once every week.

In some cases, avexitide is administered before a meal (e.g., morning meal, evening meal, or other main meals of the day). For example, avexitide can be administered before the morning meal. In some cases, avexitide is administered at least about 30 minutes to about 90 minutes (e.g., at least about 30 minutes, at least about 60 minutes, or at least about 90 minutes) before a meal (e.g., morning meal, evening meal, or other main meals of the day). For example, in some cases, avexitide is administered (e.g., subcutaneously) at least 60 minutes before a morning meal. In some cases, avexitide is administered subcutaneously once daily at least 60 minutes before a morning meal. For example, avexitide can be administered subcutaneously at an amount of 90 mg once daily at least 60 minutes before a morning meal.

As provided herein avexitide can be administered for about 1 day to about 48 weeks or more. For example, avexitide can be administered for about 4 weeks, 8 weeks, 12 weeks, 16 weeks, 20 weeks, 24 weeks, 28 weeks, 32 weeks, 36 weeks, 40 weeks, 44 weeks, 48 weeks, or more.

The dosages and dosage ranges described above are exemplary adult doses and may vary depending upon the age and weight of a subject, as would be known by those skilled in the pharmaceutical arts. It will be appreciated that in some embodiments, dosage may be increased or decreased during the course of treatment. For example, some physicians may desire to treat with a low or initiating (starting) dose, escalate to an increased dose if the initiating dose does not provide sufficient therapeutic benefit, and maintain the initiating dose if the initiating dose provides sufficient therapeutic benefit.

### Pharmaceutical Compositions

The methods described herein include the use of pharmaceutical compositions comprising avexitide as an active ingredient. The pharmaceutical compositions are also referred to herein as avexitide formulations. In some embodiments, provided herein are buffered liquid formulations comprising avexitide.

In some embodiments, the avexitide formulations comprises avexitide at a concentration of about 30-250 mg/mL (e.g., about 30-200 mg/mL, about 30-180 mg/mL, about 30-150 mg/mL, about 30-120 mg/mL, about 30-100 mg/mL, about 40-200 mg/mL, about 40-180 mg/mL, about 40-150 mg/mL, about 40-120 mg/mL, about 40-100 mg/mL, about 50-200 mg/mL, about 50-180 mg/mL, about 50-150 mg/mL, about 50-120 mg/mL, about 50-100 mg/mL, about 60-200 mg/mL, about 60-180 mg/mL, about 60-150 mg/mL, about 60-120 mg/mL, about 60-100 mg/mL, about 70-200 mg/mL, about 70-180 mg/mL, about 70-150 mg/mL, about 70-120 mg/mL, about 70-100 mg/mL, about 80-200 mg/mL, about 80-180 mg/mL, about 80-150 mg/mL, about 80-120 mg/mL, about 80-100 mg/mL, about 90-200 mg/mL, about 90-180 mg/mL, about 90-150 mg/mL, about 90-120 mg/mL, about 90-110 mg/mL, or about 90-100 mg/mL). In some embodiments, the avexitide formulations comprises avexitide at a concentration of about 30 mg/mL, about 40 mg/mL, about 50 mg/mL, about 60 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 140 mg/mL, about 150 mg/mL, about 160 mg/mL, about 170 mg/mL, about 180 mg/mL, about 200 mg/mL, about 220 mg/mL, about 220 mg/mL, about 230 mg/mL, about 240 mg/mL, or about 250 mg/mL.

As described herein, in some embodiments, avexitide is in the form of avexitide acetate. In some embodiments, avexitide acetate compositions comprise about 1.5% to about 6.5% acetate (e.g., about 1.5%, 2.0%, 2.5%, 3.0%, 3.5%, 4.0%, 4.5%, 5.0%, 5.5%, 6.0%, or 6.5% acetate). In some embodiments, avexitide is in the form of avexitide monoacetate. In some embodiments, avexitide is in the form of avexitide diacetate. In some embodiments, avexitide is in the form of avexitide triacetate. In some embodiments, avexitide is in the form of avexitide tetraacetate. In some embodiments, avexitide is in the form of avexitide pentaacetate. In some embodiments, avexitide is in the form of avexitide hexaacetate. In some embodiments, avexitide is in the form of avexitide heptaacetate. In some embodiments, avexitide acetate is a mixture of avexitide monoacetate, avexitide diacetate, avexitide triacetate, avexitide tetraacetate, avexitide pentaacetate, and/or avexitide hexaacetate. In some embodiments, avexitide is in the form of avexitide heptaacetate. In some embodiments, avexitide acetate is a mixture of avexitide monoacetate, avexitide diacetate, avexitide triacetate, avexitide tetraacetate, avexitide pentaacetate, avexitide hexaacetate, and/or avexitide heptaacetate. In some embodiments, avexitide acetate compositions comprise primarily avexitide diacetate and/or avexitide triacetate. In some embodiments, avexitide acetate compositions comprise at least about 30%, 35%, 40%, 45%, 50%, 55%, 60%, 65%, 70%, 75%, 80%, 85%, 90%, or 95% avexitide diacetate and/or avexitide triacetate.

In some embodiments, the formulation comprises avexitide or a pharmaceutically acceptable salt thereof in a physiologically acceptable buffer having a pH of about 5.0 to about 6.0. In some embodiments, the buffer is compatible with subcutaneous or intravenous administration. In some embodiments, the physiologically acceptable buffer is a buffer that results in a liquid formulation having a pH at or about physiological pH or within a relatively narrow pH range near physiological pH (for example, between about 5.0 to about 8.0). In some embodiments, the physiologically acceptable buffer has a pH of about 5.0 to about 5.5 (e.g., about 5.1, 5.2, 5.3, 5.4, or 5.5). In some embodiments, the physiologically acceptable buffer has a pH in the range of about 5.2 to about 5.7 (e.g., about 5.2, 5.3, 5.4, 5.5, 5.6, or 5.7). In some embodiments, the physiologically acceptable buffer has a pH in the range of about 5.2 to about 5.8 (e.g., about 5.2, 5.3, 5.4, 5.5, 5.6, 5.7, or 5.8). In one embodiment, the physiologically acceptable buffer has a pH of about 5.5 to about 6.0 (e.g., about 5.5, 5.6, 5.7, 5.8, 5.9, or 6.0). In some embodiments, the physiologically acceptable buffer has a pH of about 5.4. In some embodiments, the physiologically acceptable buffer has a pH of about 5.5. In some embodiments, the physiologically acceptable buffer has a pH of about 5.6. In some embodiments, the physiologically acceptable buffer has a pH of about 5.7. In some embodiments, the physiologically acceptable buffer has a pH of about 5.8.

As provided herein, the physiologically acceptable buffer can include an acetate buffer, a citrate buffer, a phosphate buffer, a histidine buffer, or a mixture thereof. In some embodiments, the physiologically acceptable buffer comprises sodium acetate, potassium acetate, trisodium citrate, magnesium citrate, potassium citrate, potassium phosphate, or a mixture thereof.

In some embodiments, the physiologically acceptable buffer comprises a buffering agent (e.g., sodium acetate) at a concentration from about 5 mM to about 30 mM, about 10 mM to about 30 mM, about 15 mM to about 30 mM, about 20 mM to about 30 mM, or about 25 mM to about 30 mM (for example, about 5 mM, about 8 mM, about 10 mM, about 12 mM, about 15 mM, about 18 mM, about 20 mM, about 22 mM, about 25 mM, about 28 mM, or about 30 mM). In some embodiments, the physiologically acceptable buffer comprises a buffering agent (e.g., sodium acetate) at a concentration of at least about 10 mM. In some embodiments, the physiologically acceptable buffer comprises an acetate buffer. In some embodiments, the buffering agent is sodium acetate. In some embodiments, the buffering agent is potassium acetate. In some embodiments, the physiologically acceptable buffer comprises an acetate buffer (e.g, sodium acetate or potassium acetate) at a concentration of about 5 mM to about 30 mM, for example, about 10 mM to about 20 mM. In some embodiments, the physiologically acceptable buffer comprises an acetate buffer (e.g., sodium acetate or potassium acetate) at a concentration of at least about 10 mM. In some embodiments, the physiologically acceptable buffer comprises sodium acetate (e.g., sodium acetate trihydrate) at a concentration from about 0.5 mg/mL to about 3 mg/mL (e.g., about 0.5 mg/mL to about 2.5 mg/mL, about 0.5 mg/mL to about 2 mg/mL, about 0.8 mg/mL to about 1.8 mg/mL, about 1 mg/mL to about 1.5 mg/mL, or about 1.3 mg/mL to about 1.4 mg/mL). For example, sodium acetate (e.g., sodium acetate trihydrate) can be present at a concentration of about 1.31 mg/mL, about 1.32 mg/mL, about 1.33 mg/mL, about 1.34 mg/mL, about 1.35 mg/mL, about 1.36 mg/mL, about 1.37 mg/mL, about 1.38 mg/mL, or about 1.39 mg/mL. In some embodiments, the physiologically acceptable buffer comprises sodium acetate (e.g., sodium acetate trihydrate) at a concentration from about 0.05% w/v to about 0.25% w/v (e.g., about 0.05% w/v to about 0.2% w/v, about 0.08% w/v to about 0.18% w/v, about 0.1% w/v to about 0.15% w/v, or about 0.13 to about 0.14% w/v). For example, sodium acetate (e.g., sodium acetate trihydrate) can be present at a concentration of about 0.1310% w/v, about 0.132% w/v, about 0.1330% w/v, about 0.134% w/v, about 0.1355% w/v, about 0.136% w/v, about 0.137% w/v, about 0.138% w/v, or about 0.139% w/v.

In some embodiments, the physiologically acceptable buffer comprises a citrate buffer. In some embodiments, the buffering agent is trisodium citrate. In some embodiments, the buffering agent is magnesium citrate. In some embodiments, the buffering agent is potassium citrate. In one embodiment, the physiologically acceptable buffer comprises the citrate buffer (for example, sodium citrate, magnesium citrate, or potassium citrate) at a concentration from about 5 mM to about 30 mM, for example, about 10 mM to about 20 mM. In some embodiments, the physiologically acceptable buffer comprises the citrate buffer (for example, sodium citrate, magnesium citrate, or potassium citrate) at a concentration of about 10 mM.

In one embodiment, the physiologically acceptable buffer comprises a phosphate buffer. In one embodiment, the physiologically acceptable buffer comprises potassium phosphate. In one embodiment, the physiologically acceptable buffer comprises potassium phosphate at a concentration from about 5 mM to about 30 mM, for example, about 10 mM to about 20 mM. In some embodiments, the physiologically acceptable buffer comprises the phosphate buffer (for example, potassium phosphate) at a concentration of about 10 mM.

As provided herein, the buffered liquid formulation can include a tonicity modifier. Suitable tonicity modifiers include, e.g., mannitol, dextrose, glycerin, lactose, sucrose, trehalose, or a mixture thereof. In some embodiments, the tonicity modifier is mannitol. Tonicity modifiers can be used to provide a liquid formulation suitable for subcutaneous administration. See, e.g., Pramanick et al., Pharma Times., Vol 45, No. 3, (2013); see also, Formulating Poorly Water Soluble Drugs, Williams, Watts, and Miller, eds., Springer Science and Business Media (2011).

In some embodiments, the tonicity modifier or combination of tonicity modifiers is present in the formulation at a concentration of about 5 mg/mL to about 75 mg/mL (e.g., about 5 mg/mL to about 60 mg/mL, about 10 mg/mL to about 50 mg/mL, about 15 mg/mL to about 40 mg/mL, about 15 mg/mL to about 30 mg/mL, about 18 mg/mL to about 25 mg/mL, or about 19 mg/mL). For example, the tonicity modifier can be present in the formulation at a concentration of about 5 mg/mL, about 8 mg/mL, about 12 mg/mL, about 15 mg/mL, about 19 mg/mL, about 22 mg/mL, or about 25 mg/mL.

Tonicity modifiers are known in the art. Suitable tonicity modifiers for the provided formulation include, e.g., mannitol, dextrose, glycerin, lactose, sucrose, trehalose or a combination thereof. In some embodiments, the tonicity modifier comprises mannitol. In some embodiments, mannitol is present at a concentration of about 5 mg/mL to about 75 mg/mL (e.g., about 5 mg/mL to about 60 mg/mL, about 10 mg/mL to about 50 mg/mL, about 15 mg/mL to about 40 mg/mL, about 15 mg/mL to about 30 mg/mL, about 18 mg/mL to about 25 mg/mL, or about 19 mg/mL). In some embodiments, mannitol is present at a concentration of about 0.5% w/v to about 30% w/v (e.g., about 10% w/v to about 2.5% w/v, about 1.5% w/v to about 2.2% w/v, or about 1.9% w/v).

In some embodiments, the buffered formulation includes two or more tonicity modifiers. For example, the buffered formulation can include two or more tonicity modifiers selected from the group consisting of mannitol, dextrose, glycerin, lactose, sucrose, and trehalose. In some embodiments, the buffered formulation comprises mannitol and at least one other tonicity modifier.

In some embodiments, formulations described herein have an osmolality of about 260 mOsm/kg to about 380 mOsm/kg (e.g., about 260, 265, 270, 275, 280, 285, 290, 295, 300, 305, 310, 315, 320, 325, 330, 335, 340, 345, 350, 355, 360, 365, 370, 375, or 380 mOsm/kg). In some embodiments, the formulation has an osmolality of at least 280 mOsm/kg (i.e., >280 mOsm/kg). In some embodiments, the formulation has an osmolality of about 280 mOsm/kg to about 380 mOsm/kg (e.g., about 280, 285, 290, 295, 300, 305, 310, 315, 320, 325, 330, 335, 340, 345, 350, 355, 360, 365, 370, 375, or 380 mOsm/kg). In some embodiments, the formulation has an osmolality of greater than about 300 mOsm/kg. In some embodiments, the formulation has an osmolality of about 300 mOsm/kg to about 380 mOsm/kg (e.g., about 300, 305, 310, 315, 320, 325, 330, 335, 340, 345, 350, 355, 360, 365, 370, 375, or 380 mOsm/kg). In some embodiments, the formulation has an osmolality of less than 300 mOsm/kg. In some embodiments, the formulation has an osmolality of about 330 mOsm/kg (e.g., about 325, 326, 327, 328, 329, 330, 331, 332, 333, 334, or 335 mOsm/kg).

In some embodiments, the formulation further comprises one or more additional excipients such as preservatives, surfactants (for example, a polysorbate or a poloxamer), or colorants (for example, pharmaceutically acceptable dyes, inorganic pigments, and natural colorants). A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc., each of which is incorporated by reference herein.

In some embodiments, the formulation comprises avexitide acetate, sodium acetate (e.g., sodium acetate trihydrate), mannitol, sodium hydroxide, glacial acetic acid, and water. For example, the formation can include 100 mg/mL avexitide acetate, 1.36 mg/mL sodium acetate trihydrate, 19 mg/mL mannitol, sodium hydroxide, glacial acetic acid, and water. In some embodiments, the avexitide acetate is a combination of avexitide monoacetate, avexitide diacetate, avexitide triacetate, avexitide tetraacetate, avexitide pentaacetate, avexitide hexaacetate, and/or avexitide heptaacetate. In some embodiments, the formulation has a pH of about 5.0 to 6.0. In some embodiments, the formulation has a pH of about 5.2 to about 5.7. In some embodiments, the formulation has a pH of about 5.4 to about 5.5. In some embodiments, the formulation has an osmolality of about 260 mOsm/kg to about 380 mOsm/kg. In some embodiments, the formulation has an osmolality of at least about 280 mOsm/kg. In some embodiments, the formulation has an osmolality of about 280 mOsm/kg to about 380 mOsm/kg. In some embodiments, the formulation has an osmolality of at least about 300 mOsm/kg. In some embodiments, the formulation has an osmolality of about 300 mOsm/kg to about 380 mOsm/kg (e.g., about 330 mOsm/kg). In some embodiments, the formulations described herein are clear to slightly opalescent. In some embodiments, the formulations described herein are colorless to pale yellow. In some embodiments, the formulations described herein are essentially free of visible particles.

The avexitide formulation as described herein can be administered in a variety of drug delivery devices, including, e.g., a single-use pre-filled syringe, a pen injector device (e.g., a disposable or reusable pen injector device), a single-use glass vial or ampule (e.g., for administration with the use of a syringe). In some embodiments, the avexitide formulation is packaged in a single-use container closure system composed of standard components used for parenterally administered drug products. In some embodiments, avexitide is supplied ready-to-use and requires no reconstitution prior to use. In some embodiments, avexitide is provided in the form of a powder (e.g. lyophilized powder) which is reconstituted into a liquid formulation prior to use.

In some embodiments, the avexitide formulation is provided as an injectable solution in a single-dose tray containing a vial of an avexitide formulation as described herein, a vial connector, a syringe, and one or more needles.

In some embodiments, each single use glass vial or container comprises about 0.2 mL to about 2 mL (e.g., about 0.4 to about 1.5 mL or about 0.5 mL to about 1.0 mL) of the avexitide solution.

In some embodiments, each dose of avexitide is administered as one injection. In some embodiments, each dose of avexitide is administered as two injections. In some embodiments, each avexitide dose is administered in a total volume ranging from about 0.5 mL to about 1.5 mL (e.g., about 0.9 mL). In some embodiments, the methods include administering about 0.9 mL of an avexitide formulation described herein which comprises avexitide acetate at a concentration of 100 mg/mL. In some embodiments, the methods include administering 0.45 mL of an avexitide formulation described herein which comprises avexitide acetate at a concentration of 100 mg/mL twice (e.g., consecutively).

### EXAMPLES

The invention is further described in the following examples, which do not limit the scope of the invention described in the claims.

### Example 1: Clinical Study Protocol for Treating Patients With PBH

This Example describes a Phase 3 clinical protocol for studying the efficacy, safety, and pharmacodynamic effect of avexitide in patients with post-bariatric hypoglycemia (PBH). In particular, the study is a Phase 3 multicenter study comprising a randomized, double-blind, placebo-controlled period that is followed by a two-part open-label extension (OLE) period. Doses are administered subcutaneously at 90 mg once per day throughout the double-blind and OLE periods of the study. Avexitide (or matching placebo, as applicable) is injected in a single-dose volume.

The initial 16-week Double-Blind study treatment period—which includes a Screening period of up to 6 weeks in duration, inclusive of a 3-week Run-in period—is designed to evaluate the efficacy and safety of 90 mg per day of avexitide (given once daily) compared to placebo in participants with PBH related to RYGB who are not adequately controlled on dietary management for reduction of hypoglycemia. Throughout the Double-Blind period, participants undergo continuous glucose monitoring via the study CGM (in blinded mode), use the study self-monitoring of blood glucose (SMBG) and electronic study diary (eDiary) devices, and follow the dietary management guidance provided for the study.

Dietary Management Guidance: At Screening Visit 1, a written description of the study dietary management guidance is provided to each participant for at-home reference. In accordance with Inclusion Criterion #1, this guidance is to be followed throughout the Screening period (including the Run-in period), the Double-Blind study treatment period, and Part A of the OLE period (i.e., throughout participants' time on blinded CGM). Participants are not required to follow this protocol-specified dietary management guidance during Part B of the OLE period, in which their CGM use is unblinded. The key points of the study dietary management guidance are summarized as follows:
Limiting dietary carbohydrates to no more than 30 g per meal or 15 g per snack (Kellogg et al., Surg Obes Relat Dis. 2008 July-August; 4(4):492-9; Bantle et al., Obes Surg. 2007 May; 17(5):592-4; Botros et al., Obes Surg. 2014 November; 24(11):1850-5; van Meijeren et al., Surg Obes Relat Dis. 2017 March; 13(3):404-410; Suhl et al., Surg Obes Relat Dis. 2017 May; 13(5):888-896)
Avoiding simple, high-glycemic index carbohydrates;
Maintaining adequate protein intake (Heber et al., J Clin Endocrinol Metab. 2010 November; 95(11):4823-43; Moize et al., Obes Surg. 2010 August; 20(8):1133-41; Mechanick et al., American Association of Clinical Endocrinologists; Obesity Society; American Society for Metabolic & Bariatric Surgery. American Association of Clinical Endocrinologists, The Obesity Society, and American Society for Metabolic & Bariatric Surgery medical guidelines for clinical practice for the perioperative nutritional, metabolic, and nonsurgical support of the bariatric surgery patient. Obesity (Silver Spring). 2009 April; 17 Suppl 1:S1-70, v. Erratum in: Obesity (Silver Spring). 2010 March; 18(3):649; Suhl et al., Surg Obes Relat Dis. 2017 May; 13(5):888-896);
Avoiding consumption of meals during the late evening;
Restricting use of alcohol to no more than 1 drink per day; and
Maintaining post-bariatric vitamin and mineral supplementation (Heber et al., J Clin Endocrinol Metab. 2010 November; 95(11):4823-43; Moize et al., Obes Surg. 2010 August; 20(8):1133-41).

The subsequent 32-week OLE period is intended to further evaluate the safety and efficacy of open-label avexitide (90 mg per day, given once daily) in participants who have completed the Double-Blind period. Throughout the 8-week initial part (Part A) of the OLE period, participants continue to use the (blinded) CGM and the SMBG and eDiary devices. They are also be required to follow the same dietary management guidance as was required in the Screening period (including the Run-in period) and the Double-Blind period.

The CGM is used in unblinded mode during the 24-week final part (Part B) of the OLE period. The SMBG and eDiary devices are not assessed during OLE Part B, and participants are not required to follow the dietary management guidance during that part of the OLE period.

The study objectives and endpoints are summarized below:

TABLE 1

| | |
|---|---|
| Objectives | Endpoints |

Primary Efficacy

| | |
|---|---|
| To evaluate the efficacy of | Composite rate of Level 2 |
| avexitide compared to placebo | hypoglycemia (as measured by |
| for reduction of Level 2 and | self-monitoring of blood glucose |
| Level 3 hypoglycemic | [SMBG]) and |
| events | Level 3 hypoglycemia (per |
| | American Diabetes Association |
| | [ADA], European |
| | Association for the |
| | Study of Diabetes [EASD]; |
| | adjudicated by independent Event |
| | Adjudication Committee [EAC]), |
| | assessed during the Double-Blind |
| | study treatment period |

Safety

| | |
|---|---|
| To evaluate the safety and | Incidence of adverse events (AEs)- |
| tolerability of avexitide | e.g., incidence of |
| | treatment-emergent |
| | AEs (TEAEs), serious AEs (SAEs), |
| | AEs of special interest (AESIs), and |
| | TEAEs leading to discontinuation- |
| | and other safety assessments (e.g., |
| | clinical laboratory results and vital |
| | sign measurements), assessed during |
| | the Double-Blind study treatment |
| | period |
| To assess the incidence of anti- | Incidence of ADAb, assessed during |
| drug antibodies (ADAb) | the Double-Blind study treatment |
| | period |

Key Secondary Efficacy (controlled for type 1 error)

| | |
|---|---|
| To further evaluate the efficacy of | Rates of the following, assessed |
| avexitide compared to placebo for | during the Double-Blind study |
| reduction of hypoglycemia | treatment period: |
| | Level 2 hypoglycemia as |
| | measured by SMBG |
| | Level 3 hypoglycemia, |
| | adjudicated by EAC |
| | Level 2 hypoglycemia as |
| | measured by CGM |

Tertiary

| | |
|---|---|
| To further evaluate the safety and | Incidence of AEs-e.g., incidence |
| tolerability of avexitide | of TEAEs, SAEs, AESIs, and TEAEs |
| over 48 weeks of continued | leading to discontinuation-and other |
| open-label treatment | safety assessments (e.g., clinical |
| | laboratory results and vital sign |
| | measurements), assessed during the |
| | OLE period |
| To further evaluate the efficacy of | Rates of the following, assessed |
| avexitide compared to placebo for | during Part A of the OLE period: |
| reduction of hypoglycemia | Level 2 hypoglycemia as |
| | measured by SMBG |
| | Level 3 hypoglycemia, |
| | adjudicated by EAC |
| | Level 2 hypoglycemia as |
| | measured by CGM |
| | Mean values for the following, as |
| | assessed throughout the Double-Blind |
| | study treatment period and the OLE |
| | period: |
| | Time below target glucose range |
| | (TBR) Level 2 as measured by |
| | CGM |
| | TBR Level 1 as measured by CGM |
| | Time in target glucose range (TIR) |
| | as measured by CGM |
| | Time above target glucose range |
| | (TAR) as measured by CGM |
| | Level 1 hypoglycemia as measured |
| | by CGM |
| | Level 1 hypoglycemia as measured |
| | by SMBG |
| | Level 2 hypoglycemia as measured |
| | by CGM |
| | Extended Level 1 hypoglycemia as |
| | measured by CGM |
| | Extended Level 2 hypoglycemia as |
| | measured by CGM |
| To evaluate participant dietary | Dietary liberalization, defined as the |
| liberalization | number of glucose excursions ≥150 |
| | mg/dL, as measured by CGM |
| To evaluate participant | QoL, as measured by change from |
| quality of life (QoL) | baseline to Week 16 and Week 48 in |
| | the following assessments: |
| | Clarke Hypoglycaemia Awareness |
| | Survey (CHAS) |
| | Gold Score |
| | Hypoglycemia Fear Survey-II |
| | (HFS-II) |
| | Short-Form (36) Health Survey |
| | version 2 (SF-36 v2) |
| | EuroQOL 5 Dimension 5-Level |
| | questionnaire (EQ-5D-5L) |
| | Functional Assessment of Chronic |
| | Illness Therapy - Fatigue scale |
| | (FACIT-Fatigue) |
| To evaluate population | Clearance (CL) parameters and |
| pharmacokinetics (PopPK) | volume of distribution (Vd) |
| of avexitide in this | parameters as determined by PopPK |
| participant population | modeling |

### Study Design

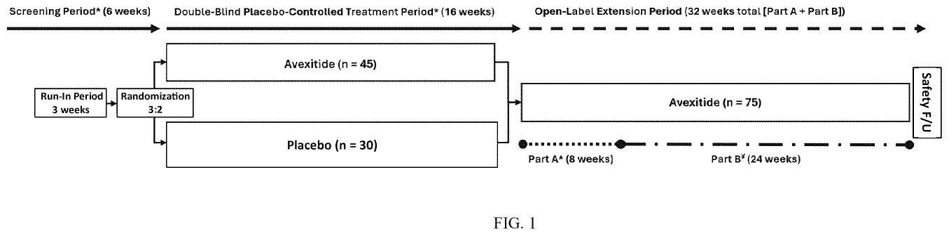

The study design schematic is presented in FIG. 1. Eligible participants must have a confirmed diagnosis of PBH related to RYGB and must have experienced at least 3 discrete hypoglycemic episodes—with ≥2 qualifying events of Level 2 or greater severity and ≥1 event adjudicated by independent EAC as Level 3 (per ADA, EASD)—during the 3-week study Run-in period, while adhering to consistent dietary management. Depending on when the participant completes Screening, the maximum anticipated time during which an individual participant may participate in this study—including the 16-week Double-Blind study treatment period, the 32-week OLE period, and the Safety Follow-up visit occurring 28 days after the last dose of study drug—is approximately 58 weeks.

### Screening Period (Including Run-In) and Double-Blind Study Period

Approximately 75 participants entering the double-blind study period are randomized and assigned in a 3:2 ratio to either the avexitide treatment group or the placebo group, respectively. The Double-Blind study treatment period comprises approximately 16 weeks of dosing by subcutaneous (SC) injection, as follows:
90 mg avexitide SC, once per day, in the morning at least 60 minutes before the morning meal; or
Placebo SC, once per day, in the morning at least 60 minutes before the morning meal.

Participants complete a 6-week Screening period prior to randomization and enrollment. Eligibility is confirmed based on the inclusion/exclusion criteria provided below.

During the Run-in period, participants adhere to the dietary management guidance summarized above and use (a) the (blinded) CGM, (b) the SMBG device provided for checking their glucose measurements when symptomatic for hypoglycemia, or when prompted by a CGM alert, and (c) the eDiary device provided for recording all hypoglycemic signs, symptoms, and outcomes, as well as other contextual information about each hypoglycemia event.

Throughout their double-blind study treatment, participants also follow the dietary management guidance provided above and use the study-provided CGM (in blinded mode), SMBG device, and eDiary device.

Participants continue into the OLE period of the study to receive open-label avexitide treatment beginning at Week 16. The OLE period comprises an additional 32 weeks of study treatment, as described below.

### Open-Label Extension Period

Participants who complete the 16-week Double-Blind period of the study per protocol enter the OLE period to receive open-label avexitide treatment beginning at Week 16. Each participant, along with the Investigators and site staff, remains blinded to the participant's previous double-blind study treatment allocation upon their entry into the OLE period.

During the entirety of the 32-week OLE period, participants are treated with open-label avexitide as follows: 90 mg avexitide SC, once per day, in the morning at least 60 minutes before the morning meal.

Part A of OLE Period: In Part A of the OLE period (8 weeks in duration), participants continue to use the study-provided CGM (in blinded mode), as well as the study SMBG and eDiary devices. Throughout Part A, participants are required to follow the same study dietary management guidance required for the Screening period (including the Run-in period) and the Double-Blind period.

Part B of OLE Period: In Part B of the OLE period (24 weeks in duration), participants use the study-provided CGM in unblinded mode. The SMBG and eDiary devices are not assessed during OLE Part B, and participants are not required to follow the dietary management guidance during that part of the OLE period.

### Study Population

The inclusion criteria include:
1. Is able to understand the purpose and risks of the study; willing and able to adhere to the scheduled visits, treatment plans, dietary requirements, laboratory tests, and procedures related to the use of the study-provided CGM device (which is used in blinded mode during the Run-in period, Double-Blind study treatment period, and Part A of the OLE Period, then in unblinded mode during Part B of the OLE period), SMBG device, and eDiary device as well as the other study evaluations and procedures; and able to provide written informed consent.
2. Is male or female, at least 18 years of age (inclusive) at the time of providing written informed consent.
3. Has body mass index (BMI) of up to 40 kg/m² and has stable body weight—i.e., not varying by >5% for at least 2 months prior to Screening—as per participant report.
4. Has undergone documented RYGB performed ≥12 months prior to Screening.
5. Has clinical diagnosis of PBH—defined as history of recurrent hypoglycemia with onset after surgery (refer to Inclusion Criterion #4 above), characterized by Whipple's triad (symptoms of hypoglycemia, concurrent low plasma glucose levels, and relief of symptoms by carbohydrate ingestion)—having ruled out other causes of hypoglycemia as per Investigator judgment.
6. Has recurrent hypoglycemia, as demonstrated by experiencing at least 3 discrete hypoglycemic episodes (as defined in under the “Efficacy Assessments—Definitions for the Primary Efficacy Endpoint” section below) during the 3-week Run-in period while adhering to consistent dietary management. At least 2 of the qualifying events must have been Level 2 or greater in severity, and at least 1 event must have been adjudicated by independent EAC as Level 3 (per ADA, EASD). Determination of Level 2 and Level 3 hypoglycemia events are based on those described in the “Efficacy Assessments—Definitions for the Primary Efficacy Endpoint” section below.
7. Must agree to consistently follow the dietary management guidance summarized above throughout the Screening period (including the Run-in period), the Double-Blind study treatment period, and Part A of the OLE period.
8. Must agree to maintain consistent exercise and/or physical activity level throughout the Screening period (including the Run-in period), the Double-Blind study treatment period, and Part A of the OLE period.

The exclusion criteria include:

### General Exclusions:

1. Has received avexitide (exendin 9-39) at any time prior to Screening Visit 1.
2. Has received another investigational drug, for any indication, within 5 half-lives of that drug prior to Screening Visit 1.
3. Has participated in another interventional clinical study within 30 days prior to Screening Visit 1.
4. Presence of gastrostomy tube (G-tube).
5. Any known or suspected allergy to one of the investigational medicinal products (avexitide or placebo) or any related product (e.g., exenatide).
6. History within 21 days prior to Screening Visit 1 of active infection considered clinically significant in the opinion of the Investigator.

### Disease-Related Exclusions

7. History or presence of insulinoma or other cause of endogenous hyperinsulinism other than PBH (e.g., insulin autoimmune hypoglycemia). Any such causes must be ruled out if clinically suspected by the Investigator.
8. Family or personal history of medullary thyroid carcinoma or multiple endocrine neoplasia syndrome type 2.
9. Family history of pancreatic cancer, or personal history of acute pancreatitis, chronic pancreatitis, or pancreatic cancer.
10. Personal history of acute hepatitis or chronic hepatitis, or signs and symptoms of liver disease except nonalcoholic fatty liver disease.
11. Active psychiatric disease or active eating disorder (e.g., uncontrolled major depressive disorder, schizophrenia, bipolar disorder, or other severe mood, anxiety, or eating disorder).
12. History of any malignancy within the 3 years prior to Screening Visit 1, except basal or squamous cell carcinoma of the skin, in situ carcinomas of the cervix, or in situ prostate cancer. Prospective participants with a history of malignancy within 3 years prior to Screening that has been treated with curative intent without recurrence may be considered on a case-by-case basis, after discussion with the Medical Monitor.
13. Uncontrolled hypertension, defined as systolic blood pressure (SBP) above or equal to 160 mmHg and/or diastolic blood pressure above or equal to 100 mmHg, on multiple assessments.
14. History of any of the following cardiovascular conditions within 12 months prior to Screening: acute myocardial infarction (MI), cerebrovascular accident (stroke), unstable angina, or hospitalization due to congestive heart failure.
15. Presence of any clinically relevant renal, hepatic, pancreatic, cardiovascular, neurological, psychiatric, hematological, pulmonary, and/or gastrointestinal abnormality which, per the judgment of the Investigator, may preclude the participant from safe completion of this study.

### Procedure-Related Exclusions

16. History of major surgery within 6 months prior to Screening.
17. History of upper GI surgery, other than RYGB (as delineated in Inclusion Criterion #4 above). Note that history of VSG with subsequent RYGB conversion may be considered on a case-by-case basis upon discussion with the Medical Monitor.

### Exclusions Based on Laboratory or Physical Examination Findings

18. Abnormal liver function, defined as transaminases (alanine transaminase [ALT], aspartate transaminase [AST]) levels>5×upper limit of normal (ULN) and/or bilirubin level>3×ULN, at Screening.
19. Renal impairment, defined as an estimated glomerular filtration rate (eGFR)<30 mL/min/1.73 m² (calculated using the Chronic Kidney Disease Epidemiology Collaboration equation), at Screening.
20. Other chemistry, hematology, physical examination, or ECG result at Screening that is deemed clinically significant in the opinion of the Investigator.

### Exclusions Based on Recent or Concomitant Medication or Drug Use

21. Current or prior use of agent(s) that may alter glucose metabolism, or promote weight loss, within 5 medication half-lives of Screening Visit 1. Such agents include, but are not limited to, the following (see Table 2): acarbose; calcium channel blockers (e.g., verapamil); diazoxide; dipeptidyl-peptidase-4 (DPP-4) inhibitors; GLP-1 agonists (e.g., exenatide, semaglutide, liraglutide); glucocorticoids (refer to the note immediately below); glucose-dependent insulinotropic polypeptide (GIP)/GLP-1 dual agonists (e.g., tirzepatide); insulin; lithium; meglitinides; metformin; pentamide; sodium-glucose-linked transporter (SGLT)-1 inhibitors; SGLT-2 inhibitors; somatostatin analogs (e.g., octreotide, pasireotide, lanreotide); sulfonylureas; and thiazolidinediones (TZDs).
   a. Note regarding glucocorticoids: Oral, intra-articular, and intravenous glucocorticoids are prohibited in this study; however, topical, intraocular, intranasal, or inhaled preparations may be acceptable on a case-by-case basis with approval by the Medical Monitor, provided that the dose and regimen remain consistent for the duration of study participation.
22. Use of drugs that interfere with the Dexcom G7 sensor within 5 half-lives of Screening Visit 1. Such drugs include acetaminophen administered at a dosage greater than 1000 mg every 6 hours, and hydroxyurea.
23. Use of drugs that affect GI motility to a degree considered significant by the Investigator. The Medical Monitor should be contacted with any questions about drugs, including laxatives, that affect GI motility to a less-than-significant degree per Investigator judgment.

### Prohibited and Restricted Medications and Procedures

Refer to Table 2 for a list of medications and procedures prohibited or restricted during study treatment.

Additionally, prospective participants are not eligible for this study if they:
Have a G-tube present;
Have previously received avexitide (exendin 9-39) at any time prior to Screening Visit 1;
Have received another investigational drug, or other study drug for any indication, within 5 half-lives of that drug prior to Screening Visit 1; or
Have participated in another interventional clinical study within 30 days before Screening Visit 1.

TABLE 2
Medications and Procedures Prohibited or Restricted during Study Participation

| | |
|---|---|
| Medications that may alter glucose | Medication(s) that are known to alter the rate of |
| metabolism or promote weight loss. These include, | nutrient transit^{b} (e.g., gastric emptying) (refer to |
| but are not limited to, the following: | footnote “b” below) |
| acarbose | Procedures that alter the gastrointestinal anatomy |
| calcium channel blockers (e.g., verapamil) | and/or may alter the rate of nutrient transit |
| diazoxide | |
| dipeptidyl-peptidase-4 | |
| (DPP-4) inhibitors | |
| GLP-1 agonists (e.g., exenatide, | |
| semaglutide, liraglutide) | |
| glucocorticoids^{a} (refer to footnote “a” | |
| below) | |
| glucose-dependent insulinotropic | |
| polypeptide (GIP)/GLP-1 dual agonists (e.g., | |
| tirzepatide) | |
| insulin | |
| lithium | |
| meglitinides | |
| metformin | |
| pentamide | |
| sodium-glucose-linked transporter (SGLT)-1 | |
| inhibitors | |
| SGLT-2 inhibitors | |
| somatostatin analogs (e.g., octreotide, | |
| pasireotide, lanreotide) | |
| sulfonylureas | |
| thiazolidinediones (TZDs) | |
| Medications that interfere with the Dexcom G7 | |
| sensor, including acetaminophen at a dosage greater | |
| than 1000 mg every 6 hours and hydroxyurea | |

### Drug Supplies

Avexitide injection, 100 mg/mL, is a sterile, clear to slightly yellow solution. The drug product composition consists of 100 mg/mL avexitide (active ingredient) in a sodium acetate buffer with mannitol as a tonicity modifier and is to be thawed prior to administration by SC injection. The study drug (i.e., avexitide or placebo) is administered by SC injection in the anterolateral aspect of the thigh or abdomen, or as deemed appropriate by the Investigator.

### Efficacy Assessments

### Definitions for the Primary Efficacy Endpoint

The primary efficacy endpoint is the composite rate of Level 2 hypoglycemia as measured by SMBG and Level 3 hypoglycemia (ADA, EASD; refer to “Level 3 Hypoglycemia Event Definition and Criteria for Classification as Severe Hypoglycemia” below)—with the latter adjudicated by independent EAC—as assessed during the Double-Blind study treatment period. Level 2 hypoglycemia is defined as glucose<54 mg/dL (3.0 mmol/L) as measured by SMBG. Level 3 hypoglycemia is defined (see “Level 3 Hypoglycemia Event Definition and Criteria for Classification as Severe Hypoglycemia” below) as a severe event characterized by altered mental and/or physical functioning that requires assistance from another person for recovery. The latter applies regardless of whether a patient actually receives external assistance.

To determine discrete episodes of Level 2 hypoglycemia from the SMBG device source data, any SMBG measurements that are <54 mg/dL (<3.0 mmol/L) within 60 minutes of the first instance of <54 mg/dL are counted as 1 distinct event. If a Level 3 hypoglycemic event occurs within the same 60-minute window of the Level 2 hypoglycemic event, as defined above, they are counted as 1 discrete event for the composite hypoglycemia event rate analysis.

### Adjudication of Level 3 Hypoglycemic Events

The categorization of a Level 3 hypoglycemic event is adjudicated by the independent EAC, which is composed of least five members. Two of the EAC members independently review each event package, and each member documents their assessment of whether or not the event classifies as Level 3 hypoglycemia as described in the EAC Charter. If the initial two reviewers disagree on the event classification, the event package is distributed to a third EAC member to review for final consensus. As described in the EAC Charter, a panel meeting may be initiated by an EAC member, the EAC Chair, or the Sponsor to further discuss an event.

A sensitivity analysis that assumes all disagreements are confirmed as Level 3 hypoglycemia, and a separate sensitivity analysis that assumes all disagreements are not Level 3 hypoglycemia, is conducted.

If a Level 3 hypoglycemic event occurs within the same 60-minute window of the Level 2 hypoglycemic event, as defined above, they are counted as 1 discrete event for the composite hypoglycemia event rate analysis.

### PBH Impact Assessment

All participants complete a self-reported PBH impact assessment. The PBH impact assessment is a self-reported questionnaire intended to assess the participant's disease burden (e.g., their diet, activities of daily living, ability to work, etc.) and healthcare resource utilization associated with PBH.

### Quality of Life Assessments

All participants complete self-reported questionnaires to include the CHAS, Gold Score, HFS-II, SF-36 v2, EQ-5D-5L, and FACIT-Fatigue.
The CHAS is an 8-item, self-administered questionnaire intended to examine the glycemic thresholds at which an individual's symptoms of hypoglycemia develop, as well as to characterize that individual's exposure to episodes of moderate and severe hypoglycemia (Clarke et al., Diabetes Care. 1995 April; 18(4):517-22.).
The Gold Score is a self-reported measurement that assesses impaired awareness of hypoglycemia. The scoring method is based on an individual's response to a single question, with responses expressed in a Likert scale (Clarke et al., Diabetes Care. 1995 April; 18(4):517-22.).
The HFS-II is a 33-item questionnaire with 2 subscales that measure 1) behaviors to avoid hypoglycemia and its negative consequences, and 2) worries about hypoglycemia and its negative consequences (Cox et al., Diabetes Care. 1987 September-October; 10(5):617-21; Gonder-Frederick et al., Diabetes Care. 2011 April; 34(4):801-6) Responses are made on a 5-point Likert scale where 0=Never and 4=Always.
The SF-36 v2 consists of 36 participant-reported outcomes. It is widely used as a QoL measurement to assess health-related limitations within 8 domains: vitality, physical functioning, bodily pain, general health perceptions, physical functioning, emotional functioning, social functioning, and mental health (Brazier et al., BMJ. 1992 Jul. 18; 305(6846):160-4).
The EQ-5D-5L questionnaire is a self-report survey that measures QoL in adults, consisting of 5 dimensions—mobility, self-care, usual activities, pain and discomfort, and anxiety and depression—each of which has 5 severity levels that are described by statements appropriate to that dimension. (EuroQol Group Association. EQ-5D-5L User Guide. Version 3.0 (updated September 2019). Available from: https://euroqol.org/wp-content/uploads/2023/11/EQ-5D-5LUserguide-23-07.pdf Accessed 4 Sep. 2024.)
The FACIT-Fatigue is a 13-item measure that assesses self-reported fatigue and its impact upon daily activities and function. The scale measures an individual's level of fatigue during their usual daily activities over the past week. The level of fatigue is measured on a 4-point Likert scale (4=not at all fatigued to 0=very much fatigued) (Webster et al., Health Qual Life Outcomes. 2003 Dec. 16; 1:79.)

### Level 3 Hypoglycemia Event Definition and Criteria for Classification as Severe Hypoglycemia

Level 3 hypoglycemia (American Diabetes Association [ADA]) denotes a severe event characterized by altered mental and/or physical functioning that requires assistance from another person for recovery. This applies regardless of whether a patient actually receives external assistance. Additionally, any adverse events (AEs) that are the result of hypoglycemia should be reviewed for potential Level 3 (severe) hypoglycemia adjudication by the independent EAC in accordance with the above guideline.

Criteria for a Level 3 Event must meet both 1 and 2 of the following:
1) Event is deemed consequent to hypoglycemia. Evidence to support the hypoglycemia state may include but is not limited to self-monitoring of blood glucose (SMBG) values, continuous glucose monitoring (CGM) values/curves, central laboratory blood glucose values or medical record-documented blood glucose values. Other contextual information that supports the hypoglycemia state may be used including but not limited to; responsiveness to carbohydrate intake to correct the event, post-prandial state, or physical activity state (i.e., exercise).
AND
2) Meets at least one of the following which indicates the event would require assistance from another person for recovery (regardless of whether or not assistance was received):
   a. Patient is unable to independently obtain rescue therapy (e.g., agents used to raise blood glucose concentrations, such as glucagon, glucose tablets, juice, food, etc.). Includes preparing, retrieving, or administering rescue therapy.
   b. Patient requires assistance to prevent injury. Includes helping place the patient in a seated or reclined position, if at risk of injury (e.g., head trauma) due to potential loss of consciousness or seizure.
   c. Patient requires assistance from a medical professional (e.g., call to medical professional or ambulance, visit to doctor or emergency room).
   d. Patient requires hospitalization.
   e. Patient experiences an altered mental and/or physical functioning defined as any one of the following neuroglycopenic signs/symptoms:
      i. Cognitive deterioration, abnormal mentation, impaired judgement
      ii. Personality/behavior change, bizarre behavior
      iii. Confusion, amnesia, delirium
      iv. Vision change, blurred vision, double vision
      v. Difficulty speaking, slurred speech, nonsensical speech
      vi. Coordination difficulties, ataxia
      vii. Focal or general motor deficit
      viii. Stupor, presyncope
      ix. Loss of consciousness
      x. Convulsions, generalized, or focal seizures
      xi. Coma/obtundation
      xii. Stroke-like signs or symptoms, including cognitive, speech, and/or motor deficits
      xiii. Note: Any sign/symptom reported by the patient that is not specifically included in the list above but appears to be similar (example, patient reports “passed out” but did not report “loss of consciousness”) may qualify at the discretion of the adjudication committee.
   f. Patient is involved in an accident (e.g., motor vehicle accidents, accidents at home, accidents in the workplace).
   g. Patient experiences an injury (due to falls, motor vehicle accidents, loss of consciousness/syncope, or seizure)

### Pharmacokinetic and ADAb Assessments

Plasma samples for sparse PK and for measurement of ADAb levels are collected at the specified visits. PopPK modeling is used to determine CL and Vd parameters in this participant population. In the instance of a moderate-to-severe injection site AE wherein a local hypersensitivity reaction is suspected, unscheduled blood samples for PK and ADAb are collected as close as possible to the onset of the event, at the resolution of the event, and 30 days±5 days following the event. For participants who test positive for ADAb, ADAb and concomitant PK testing is repeated by scheduled or unscheduled assessments until ADAb levels return to baseline values.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A formulation comprising avexitide or a pharmaceutically acceptable salt thereof at a concentration between about 90 mg/mL and 110 mg/mL, sodium acetate trihydrate at a concentration between about 1 mg/mL and about 1.5 mg/mL, and mannitol at a concentration between about 18 mg/mL and about 25 mg/mL,
wherein the formulation has a pH of about 5.2 to about 5.7, and
wherein the formulation has an osmolality of about 300 mOsm/kg to about 380 mOsm/kg.

2. The formulation of claim 1, wherein the formulation comprises the avexitide or the pharmaceutically acceptable salt thereof at a concentration of about 100 mg/mL.

3. The formulation of claim 1, wherein the formulation comprises sodium acetate trihydrate at a concentration of about 1.36 mg/mL.

4. The formulation of claim 1, wherein the formulation comprises mannitol at a concentration of about 19 mg/mL.

5. The formulation of claim 1, wherein the formulation has a pH of about 5.4 or about 5.5.

6. The formulation of claim 1, wherein the formulation has an osmolality of about 330 mOsm/kg.

7. A method of treating post-bariatric hypoglycemia (PBH) in a subject in need thereof, comprising administering to the subject the formulation of claim 1, and wherein the method reduces a composite rate of Level 2 hypoglycemia events and Level 3 hypoglycemia events in the subject relative to baseline.

8. The method of claim 7, wherein the method comprises administering the formulation at a dose of 50 mg to 150 mg of the avexitide or the pharmaceutically acceptable salt thereof.

9. The method of claim 7, wherein the subject has undergone bariatric surgery at least 12 months prior to administration.

10. The method of claim 9, wherein the bariatric surgery is Roux-en-Y gastric bypass or vertical sleeve gastrectomy.

11. The method of claim 7, wherein the subject has experienced at least three hypoglycemic events during a 3-week window prior to administration, wherein at least two of the at least three hypoglycemic events are Level 2 or greater in severity, and wherein at least one of the at least three hypoglycemic events is Level 3.

12. The method of claim 7, wherein the subject has refractory PBH.

13. The method of claim 7, wherein Level 2 hypoglycemia events are measured by self-monitoring of blood glucose (SMBG) and/or continued glucose monitoring (CGM).

14. The method of claim 7, wherein the method reduces the composite rate of Level 2 hypoglycemia events and Level 3 hypoglycemia events in the subject relative to baseline by at least about 10%.

15. The method of claim 7, wherein the method further reduces Level 1 hypoglycemia events in the subject relative to baseline and/or dietary liberalization in the subject relative to baseline.

16. The method of claim 7, wherein the method reduces the percent time with glucose levels less than about 70 mg/dL for the subject relative to baseline.

17. The method of claim 7, wherein the method reduces the percent time with glucose levels less than about 54 mg/dL for the subject relative to baseline.

18. The method of claim 8, wherein the method comprises administering about 80 mg to about 100 mg of the avexitide or the pharmaceutically acceptable salt thereof per day.

19. The method of claim 18, wherein the avexitide or the pharmaceutically acceptable salt thereof is administered once per day at a dose of about 90 mg.

20. The method of claim 7, wherein the avexitide or the pharmaceutically acceptable salt thereof is administered at least about 30 minutes to about 90 minutes before a meal.

21. The method of claim 7, wherein the avexitide or the pharmaceutically acceptable salt thereof is administered subcutaneously in a total volume of about 0.5 mL to about 1.5 mL.

22. The method of claim 21, wherein the avexitide or the pharmaceutically acceptable salt thereof is administered subcutaneously in a total volume of about 0.9 mL.
